(19) United States
(12) Patent Application Publication
(10) Pub. No.: US 2010/0028901 A1
(43) Pub. Date: Feb. 4, 2010

(54) METHOD FOR DIAGNOSING IN VITRO OR EX VIVO PSYCHIATRIC DISORDERS AND/OR INTESTINAL DYSBIOSES

(75) Inventors: Celine Chery, Nancy (FR); Emmanuelle Lefebvre, Vandoeuvre (FR); Marc Merten, Einville-au-Jard (FR)

Correspondence Address: YOUNG & THOMPSON, 209 Madison Street, Suite 500, Alexandria, VA, 22314, US

(73) Assignee: AURE, Nancy (FR)

(21) Appl. No.: 12/297,197

(22) PCT Filed: Apr. 13, 2007
(86) PCT No.: PCT/FR2007/000626

§ 371 Date: Oct. 15, 2009

(30) Foreign Application Priority Data
Apr. 14, 2006 (FR) 0603346

(51) Int. Cl. G01N 33/53; C07K 16/00; C12N 5/12

(52) U.S. Cl. 435/7.1; 530/388.9; 530/389.8; 435/346

(57) ABSTRACT

The invention relates to the use of at least one of the following Formula (I) compounds:

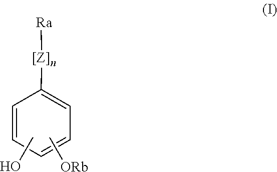

wherein
n=1,
Ra notably represents —COOH,
Rb notably represents —CH₃—,
Z notably represents —CH₂—CHOH—,
wherein the compound may be in the form of salts or, if at least one asymmetric carbon atom is present, in the form of isolated isomers or as a racemic mixture,

as a physiological marker for carrying out a method for the in vitro or ex vivo diagnosis of psychiatric disorders and/or intestinal dysbioses.

This invention relates to a new biological marker for the diagnosis of psychiatric disorders and/or intestinal dysbioses.

More particularly the invention relates to a diagnostic method, notably in vitro or ex vivo, for psychiatric disorders, notably autism, and/or intestinal dysbioses.

More particularly the invention relates to a diagnostic method, notably in vitro or ex vivo, for autism or autism-linked intestinal dysbioses.

Autism in children is a heterogeneous psychiatric disease when considered from the point of view of pathogenicity mechanisms. This pathology, also called Kanner's syndrome, usually crops up in the first years of childhood, when the child is one, two or three years old. It is characterized by language disorders, an inability to socialize, by ritual and repetitive behaviour, and finally by a mental retardation of variable intensity.

A genetic diathesis for this disease is highly suspected. However a growing amount of research work links the malfunction of the brain to a metabolic unbalance whose origin could be found in the intestines. An unbalance in the intestinal flora, also called intestinal dysbiosis, is very often to be found with persons suffering from psychiatric diseases.

Diagnosis for psychiatric disorders, notably for autism, is usually based upon close clinical examination of a given individual. Diagnosis through observation is made more complex by the fact that, on the one hand, a number of various disorders of the central nervous system may also result in symptoms which are quite comparable to those of autism, and on the other hand these tedious observations made on children must be repeated and prolonged in time, and be carried out by experienced specialists in a hospital.

Patent application EP 0 979 828 relates to a method for the diagnosis of autism comprising the detection of peptides having the formula pyroGlu-Trp-GlyNH2, Gly-Ser-Glu-Asn and pyroGlu-Glu-Asp-Ser, in a biological fluid such as blood or urine, using appropriate methods such as HPLC or immunoprecipitation with the help of antibodies against the above-mentioned peptides. This application relies on the assumption that a high rate of the above-mentioned metabolites in autistic patients may be related to an infection by fungi.

U.S. Pat. No. 5,686,311 discloses a method for the diagnosis of autism which is based on the determination of a series of metabolites which 1) may have an action on the Krebs Cycle, or 2) have a bacterial origin, such as citramalic acid, citric acid, tartaric acid, furan-2,5-dicarboxylic acid, 3-oxo-glutaric acid, dihydroxyphenylpropionic acid, carboxycitric acid, 5-hydroxymethyl-2-furoic acid, carboxyfuranglycine, phenylcarboxylic acid and arabinose. These compounds are detected by GC/MS in biological fluids (urine, blood, saliva and cerebrospinal fluid) from autistic patients, and their rates are compared with those detected in samples from healthy patients. However the use of these metabolites as biological markers does not always seem to allow one to make a diagnosis of psychiatrical disorders without resorting to the study of clinical symptoms.

Reichelt et al. (Biol. Psychiatry, 1986, 21 (13)1279-90; Adv. Biochem. Psychopharmacol., 1981, 28, p. 627-43; Med. Hypothese, 1995, 45 (5), p. 498-502) have detected a group of peptides in autistic children, and have purified some of these peptides as present in the urine. In the aim of identifying patients among whom the autism syndrome would be caused by a common pathogenous mechanism, these peptides such as gluten and caseine are much sought after. The above-cited peptides seem to have been detected in some patients only, and not in all cases of autism.

Various publications relate to the study of autism through measurement of the rate of a major dopaminergic metabolite—homovanilic acid (HVA)—or of a metabolite which is derived from norepinephrine—vanillylmandelic acid (VMA)—in a biological fluid.

These publications specify that autism seems to be linked with a neurobiological disorder, but show contradictory results.

As an example one may cite the publication of Robert T. Schultz et al. (Charney, D. S. and Nestler, E. J. The neurobiology of Mental Illness (2nd edition), Oxford University Press), in which it is said that the HVA level in the cerebrospinal fluid of autistic patients is either increased or lowered, or else identical to that of healthy individuals; by the same account some studies of the HVA urinary level report that these levels are similar whether the subjects are autistic or healthy.

In the publication by Christopher J. McDougle (J Clin Psychiatry 2005; 66 (suppl 10)), it is also stated that the HVA urinary level among autistic individuals does not differ from that of healthy individuals.

In the same way, in the publication by Minderaa R B (Noradrenergic and adrenergic functioning in autism, Biol Psychiatry. 1994 Aug. 15; 36(4):237-41. PMID: 79866888), the urinary rate of vanillylmandelic acid among healthy and autistic individuals is measured (Table 5 of that publication). It is shown that autistic individuals have a urinary vanillylmandelic acid rate which is identical to that of control subjects.

To this date prior art methods do not yield a satisfying solution to the problem of a rapid and secure diagnosis of autism with the help of biological markers, whereas it is known that any single one of them is intrinsically insufficient for discriminating between a physiological state and a pathological condition.

One of the aims of the invention is to provide a new biological marker which is sensitive, reliable and rapid to use, for the diagnosis of psychiatric disorders, notably autism and/or intestinal dysbioses.

Another aim of the invention is to provide a diagnostic kit for psychiatric disorders, notably autism and/or intestinal dysbioses.

One of the aims of the invention is to provide monoclonal and polyclonal antibodies as well as hybridoma which may produce these antibodies, and which may be used in the diagnostic kit or as antagonists for the treatment of psychiatric disorders and/or intestinal dysbioses.

The invention relates to the use of at least one of the following Formula (I) compounds

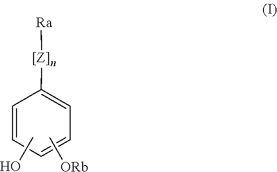

wherein
n=0 or 1,
Ra represents —CH₂OH, —COOH, —NR₁R₂, with R₁ and R₂ independently representing a hydrogen atom or a C₁-C₃ alkyl group,
Rb represents a C₁-C₃ alkyl group, preferably a —CH₃ group,
Z represents —CH₂—Y—, with Y representing a C₁-C₂ hydrocarbon chain, which may be substituted by a OH group, wherein said chain may comprise a —NH— and/or —CO— group,
wherein said compounds may be in the form of salts or, when at least one asymmetric carbon atom is present, in the form of isolated isomers or as a racemic mixture,
wherein said compounds differ from vanillyllactic acid, vanillylpyruvic acid and 3-methoxy-4-hydroxyphenylglycol,

as a physiological marker for the implementation of an in vitro or ex vivo method for the diagnosis of psychiatric disorders and/or intestinal dysbioses.

In the invention it has been surprisingly found that in patients with psychiatric disorders the peak value of at least one of the above-defined compounds is significantly above the value which is obtained for a healthy individual.

It has also been surprisingly found that the presence of at least one of the hereabove-defined compounds in a patient's biological sample allows one to advantageously diagnose autism or intestinal dysbioses which are associated with autism.

In other words, and in an advantageous manner, the inventive compounds, being specific for the diagnosis of psychiatric disorders (which may be associated with dysbioses), do not allow one to diagnose dysbioses which are not associated with psychiatric disorders.

By ‘physiological marker’ is meant a biological marker which makes it possible to implement an in vitro or ex vivo diagnostic method.

Salts are meant to denote pharmacologically compatible salts such as ammonium, sodium, potassium, calcium, or magnesium salts.

‘Psychiatric disorders’, as used in this invention, refers to psychiatric or neurological disorders. Neurological disorders pertain to disorders of the nervous system. Psychiatric disorders are mental or emotional disorders or dysfunctions. These disorders are identified and classified in Diagnostic and Statistical Manual of Mental Disorders, Fourth Edition, Revised: DSM-IV-TRX, Washington, D.C.: American Psychiatric Association; 2000.

The term ‘intestinal dysbiosis’ defines an unbalance of the intestinal flora which is usually observed among people who suffer from autistic type psychiatric disorders.

According to an advantageous embodiment the invention relates to the use, as hereabove defined, of at least one of the following Formula (I′) compounds:

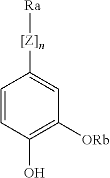

wherein:
n=0 or 1,
Ra represents —CH₂OH, —COOH, —NR₁R₂, wherein R₁ and R₂ independently represent a hydrogen atom or a C₁-C₃ alkyl group,
Rb represents a C₁-C₃ alkyl group, preferably a —CH₃ group,
Z represents —CH₂—Y—, wherein Y represents a C₁-C₂ hydrocarbon chain, which may be substituted with a OH group, wherein said chain may comprise a —NH— and/or —CO— group,
wherein said compounds may be in the form of salts or, when at least one asymmetric carbon atom is present, in the form of isolated isomers or as a racemic mixture,
wherein said compounds differ from vanillyllactic acid, vanillylpyruvic acid or 3-methoxy-4-hydroxyphenylglycol,

as a physiological marker for the implementation of an in vitro or ex vivo diagnostic method for psychiatric disorders and/or intestinal dysbioses.

According to another advantageous embodiment the invention relates to the use, such as hereabove defined, of at least one of the following Formula (I) compounds

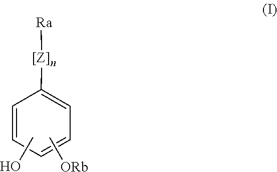

wherein:
n=0 or 1,
Ra represents —COOH,
Rb represents a C₁-C₃ alkyl group, preferably a —CH₃ group,
Z represents —CH₂—Y—, wherein Y represents a C₁-C₂ hydrocarbon chain, which may be substituted with an OH group, wherein said chain may comprise a —NH— and/or —CO— group,
wherein said compounds may be in the form of salts or, when at least one asymmetric carbon atom is present, in the form of isolated isomers or as a racemic mixture,
wherein said compounds differ from vanillyllactic acid or vanillylpyruvic acid.

According to another advantageous embodiment the invention relates to the use, such as hereabove defined, of at least one of the following Formula (II) compounds

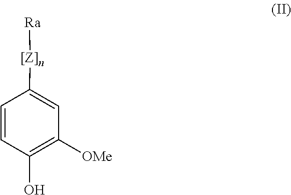

wherein n, Ra, and Z are such as hereabove defined.

According to another embodiment the invention relates to the use, such as hereabove defined, of at least one of the Formula (I) or (II) compounds, wherein:
n=0 or 1,
Ra represents —COOH,
Z represents —CH₂—CHOH—, —CH₂—CH₂—, —CO—NH—CH₂—,
wherein said compounds differ from vanillyllactic acid.

According to an advantageous embodiment the invention relates to the use, such as hereabove defined, of at least one of the following Formula (I) or (II) compounds, wherein:
n=1,
Ra represents —COOH,
Rb represents —CH₃—,
Z represents —CH₂—CHOH—,
wherein said compounds differ from vanillyllactic acid.

According to an advantageous embodiment of the invention the Formula (II) compound is chosen among one of the following compounds:

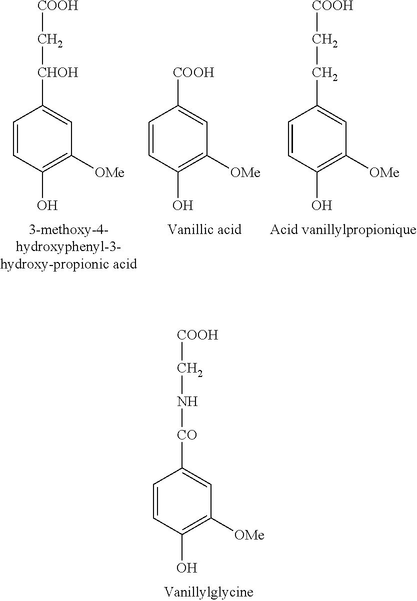

According to another advantageous embodiment of the invention the Formula (II) compound which is used is 3-methoxy-4-hydroxyphenyl-3-hydroxy-propionic acid, which corresponds to the following Formula:

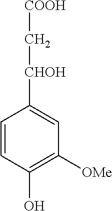

The compound as hereabove defined is 3-methoxy-4-hydroxyphenyl-3-hydroxy-propionic acid, also called vanillyl-3OH-propionic acid or vanillylhydracrylic acid. This compound is an intermediate product of one of the bacterial degradation pathways of ferulic acid (as derived from food polyphenols).

In the invention it has been surprisingly found that with patients suffering from psychiatric disorders the peak value of 3-methoxy-4-hydroxyphenyl-3-hydroxy-propionic acid is significantly above the value which is obtained for a healthy individual. The significant levels of said compound, as evidenced by appropriate methods, notably GC/MS, with all patients under examination, allow one to consider said compound as a sensitive and reliable marker for the diagnosis of autism and other psychiatric disorders.

According to another advantageous embodiment of the invention the Formula (II) compound which is used is vanillic acid, and corresponds to the following Formula:

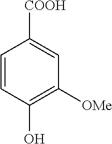

In the invention it has been surprisingly found that with patients suffering from psychiatric disorders the peak value of vanillic acid is significantly above the value which is obtained for a healthy individual.

The compounds may be used alone or as a mixture, for instance Vanillylpropionic acid (VPA) as a mixture with Vanillylglycine (VG); or Vanillylpropionic acid (VPA) as a mixture with Vanillic acid (VA); or Vanillylpropionic acid (VPA) as a mixture with Vanillylglycine (VG) and Vanillic acid (VA); or again Vanillic acid (VA) as a mixture with Vanillylglycine (VG).

The psychiatric disorders as involved in the invention belong to the group comprising autism, development retardation, neurodegenerative diseases such as Alzheimer's disease, or Parkinson's disease.

According to another advantageous embodiment the psychiatric disease as involved in the invention is autism.

Advantageously the psychiatric disorder as implied in the invention is autism when linked to intestinal dysbiosis.

The invention also relates to a diagnostic and/or prognosis method, notably in vitro or ex vivo, for psychiatric disorders and/or intestinal dysbioses, characterised in that it includes a determination step for the variation in the amount of at least one of the above-defined compounds, as present in a biological sample which is taken from a patient, in relation to the amount of this (these) same compound(s) as present in a biological sample taken from a healthy individual.

The diagnosis is positive if the variation in the amount, for at least one of the compounds, for instance in a urinary sample, and notably for 3-methoxy-4-hydroxyphenyl-3-hydroxy-propionic acid, is superior or equal to 20 micrograms per milligram of creatinine, preferentially from 20 to 180 micrograms per milligram of creatinine.

According to an advantageous embodiment the invention relates to a method such as hereabove defined, wherein the determination—with an appropriate method—of an increase in the amount of at least one of the above-defined compounds, as present in a biological sample taken from a patient, in relation to the amount of this (these) same compound(s), as present in a biological sample taken from a healthy individual, corresponds to the diagnosis of autism and/or intestinal dysbioses.

According to another advantageous embodiment the invention relates to the above-defined method wherein the amount of at least one of the above-defined compounds, as present in a biological sample which is taken from a patient, and the amount of this (these) same compound(s), as present in a biological sample taken from a healthy individual, are measured by chromatography, by spectrophotometry, by an immunological method, notably with an ELISA assay, or an immunonephelemetric method, and preferentially by gas chromatography, combined with mass spectrometry (GC/MS).

These methods are known and used by art specialists, and are successfully applied for the routine analysis of biological samples (serum, urine, blood, etc.) in order to assay a number of analytes such as proteins, peptides, natural steroids or drugs. These methods are referenced in the work by P. Tijssen <<Practice and Theory of Enzyme Immunoassays>>, Elsevier, Amsterdam 1985. However the main benefit of the GC/MS process is that it is simple to handle, it only needs a short time interval for analysis, and—above all—it is very specific and has a high detection sensitivity, allowing a finer characterization of said compound in biological samples, as compared with the other methods as known by art specialists.

In the invention the analysis and quantification of 3-methoxy-4-hydroxyphenyl-3-hydroxy-propionic acid by GC/MS may be carried out from characteristic ionic fragments having a m/z ratio of 297 and 428.

According to an advantageous embodiment of the invention, in the method such as hereabove defined, the determination of an increase in the amount of said compound having the Formula:

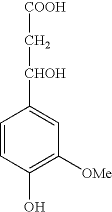

as present in a biological sample taken from a patient, as compared with the amount of the same compound which is present in a biological sample when taken from a healthy individual, corresponds to the diagnosis of autism and/or intestinal dysbioses.

According to an advantageous embodiment the biological sample which is involved in the invention in chosen among urine, blood, saliva, the cerebrospinal fluid or stools.

According to another advantageous embodiment the invention relates to the method as hereabove defined, wherein the amount of at least one of the above-defined compounds, notably the amount of the compound 3-methoxy-4-hydroxyphenyl-3-hydroxy-propionic acid, as present in an urine sample taken from a patient, is superior or equal to 20 micrograms per milligram of creatinine, and notably from 20 to 180 micrograms per milligram of creatinine.

The invention relates to monoclonal or polyclonal antibodies as directed against at least one of the above-defined compounds, and particularly against 3-methoxy-4-hydroxyphenyl-3-hydroxy-propionic acid.

The monoclonal antibodies may be obtained by immunization of mice with one of the above-defined compounds, for example 3-methoxy-4-hydroxyphenyl-3-hydroxy-propionic acid, combined with a coupling protein or peptide according to conventional techniques as known by art specialists. The immunocytes which are taken after immunization are fused with a murine myeloma which is defective for hypoxanthine guanine phosphoribosyl transferase. A selective medium allows the hybridoma to survive, and the selection of the hybridoma secreting the specific antibody against 3-methoxy-4-hydroxyphenyl-3-hydroxy-propionic acid is carried out following conventional immunological techniques.

The invention also relates to a diagnostic kit for psychiatric disorders and/or intestinal dysbioses, comprising:
one or several reagents, notably an antibody against at least one of the above-defined compounds, and which may detect, in a biological sample, the presence of at least one of the above-defined compounds,
as the case may be, a control sample containing at least one of the compounds, in a predetermined amount which is indicative of a positive diagnosis for psychiatric disorders and/or intestinal dysbioses,
as the case may be, a control sample containing at least one of the compounds, in a predetermined amount which is indicative of a negative diagnosis for psychiatric disorders and/or intestinal dysbioses.

The method which is used for manufacturing the kit is of the competitive ELISA type. It is based on the competition between one of the above-defined compounds, for instance 3-methoxy-4-hydroxyphenyl-3-hydroxy-propionic acid, conjugated with peroxydase or a fluorochrome, or a radiolabelled conjugate, and 3-methoxy-4-hydroxyphenyl-3-hydroxy-propionic acid as present in the sample, and this for a limited number of binding sites (absorbed antibodies). The amount of bound conjugate is detected by counting the radioactivity or by fluorimetry, or by the addition of a chromogenic or luminescent substrate, followed by spectrometric detection. Quantitative results are obtained by comparison with a standard interval which may be found in the kit. Intensity readings are inversely proportional to the amount of 3-methoxy-4-hydroxyphenyl-3-hydroxy-propionic acid which is present in the standard or the sample.

The invention relates to hybridoma which may produce monoclonal antibodies such as hereabove defined, particularly monoclonal antibodies against 3-methoxy-4-hydroxyphenyl-3-hydroxy-propionic acid.

The invention relates to the use of antagonists of at least one of the above-defined compounds, for the treatment of psychiatric disorders and/or intestinal dysbioses.

Advantageously the antagonists are chosen among polyclonal or monoclonal antibodies against the above-described compounds.

### DESCRIPTION OF FIGURES

FIG. 1:

FIG. 1 corresponds to the chromatogram as obtained by gas chromatography, combined with mass spectrometry (GC/MS) after analysis of a urine sample from a healthy control. This Figure illustrates the concentrations of the various trimethylsilyl derived compounds (TMS) wherein peaks are identified, such as: A, lactic acid; B, urea; C, inner standard; D, 2-oxoglutaric acid; E, hippuric acid; F, 3-methoxy-4-hydroxyphenyl-3-hydroxy-propionic acid; G, citric acid.

FIG. 2:

FIG. 2 corresponds to the chromatogram as obtained by gas chromatography, combined with mass spectrometry (GC/MS) after analysis of a urine sample from an autistic child. This Figure illustrates the concentrations of the various trimethylsilyl derived compounds (TMS) wherein peaks are identified, such as: A, lactic acid; B, urea; C, inner standard; D, 2-oxoglutaric acid; E, hippuric acid; F, 3-methoxy-4-hydroxyphenyl-3-hydroxy-propionic acid; G, citric acid.

FIG. 3:

FIG. 3 corresponds to the chromatogram as obtained by gas chromatography, combined with mass spectrometry (GC/MS), allowing one to characterize the mass spectrum of the compound which is identified as the TMS derivative of 3-methoxy-4-hydroxyphenyl-3-hydroxy-propionic acid found in an autistic patient. The x axis corresponds to the mass in Dalton of the ionic fragments. Ionic fragments 297 and 428 allow one to identify Compound A, or 3-methoxy-4-hydroxyphenyl-3-hydroxy-propionic acid.

FIG. 4:

FIG. 4 corresponds to the comparative bar chart pertaining to the amount of the TMS derivative of 3-methoxy-4-hydroxyphenyl-3-hydroxy-propionic acid, respectively between 27 healthy controls (A) and 27 autistic patients (B). The x axis corresponds to the amount of 3-methoxy-4-hydroxyphenyl-3-hydroxy-propionic acid in μg/mg creatinine. The median values are represented, as well as their 95% percentiles. The amount of said compound in autistic patients is superior or equal to 20 μg/mg creatinine, and with healthy controls it it comprised from 0 to 15 μg/mg creatinine. Moreover data from chromatographic analyses show that the surface ratio of the amount of said compound with autistic patients is superior or equal to 0.1, and that with healthy controls it is from 0 to 0.07 (results not shown). The Mann and Whitney test reveals a significant difference with an Ucal figure of 3.2.

FIG. 5:

FIG. 5 corresponds to the comparative bar chart relating to the amount of the TMS derivative of 3-methoxy-4-hydroxyphenyl-3-hydroxy-propionic acid between 62 healthy controls (A) and 45 patients suffering from psychiatric pathologies which are characterized by a severe development retardation and are not autistic (B). The x axis corresponds to the amount of 3-methoxy-4-hydroxyphenyl-3-hydroxy-propionic acid in μg/mg creatinine. The median values are represented as well as their 95% percentiles. The amount of said compound with patients suffering from various non autistic neuropathies is superior or equal to 20 μg/mg creatinine, and with healthy controls it is from 0 to 15 μg/mg creatinine. Moreover data from chromatographic analyses show that the surface ratio between rates of said compound for patients with various non autistic neuropathies and for healthy controls is from 0 to 0.07 for healthy controls and superior or equal to 0.1 for patients from Group (B) (results not shown). The Mann and Whitney test reveals a significant difference with a Ucal figure of 4.74.

FIG. 6:

FIG. 6 shows the bar chart pertaining to the amount of TMS derivative of vanillylglycine (VG) respectively between 27 healthy controls (A) and 27 autistic patients (B). The x axis corresponds to the surface ratio of the amount of vanillylglycine. Median values are represented, as well as their 95% percentiles. The value of the median with autistic patients (B) is 0.2 and the value of the median with healthy controls (A) is 0.04. For vanillylglycine (VG), the surface ratio of the amount of said compound with autistic patients is from 0.12 to 0.72, and with healthy controls it it from 0 to 0.1. The Mann and Whitney test reveals a significant difference with an Ucal figure of 3.6.

Vanillic acid (VA) and vanillylpropionic acid (VPA) have also been tested, and have shown a surface ratio with autistic patients which is above that of healthy individuals.

FIG. 7:

FIG. 7 shows the bar chart pertaining to the amount of TMS derivative of vanillic acid (VA) respectively for 38 control subjects (A) and 40 autistic patients (B). The x axis corresponds to the surface ratio of the amount of vanillic acid. The median values are represented as well as their 95% percentiles. The median value with autistic patients (B) is 0.44 and the median value with healthy controls (A) is 0.124. Moreover data from chromatographic analyses show that the surface ratio, in relation to the inner standard, of the amount of said compound with autistic patients is from 0.16 to 1.07, and that it is from 0.07 to 0.45 with healthy controls. The Mann and Whitney test reveals a significant difference with an Ucal figure of 3.63.

FIG. 8:

FIG. 8 shows the bar chart pertaining to the amount of TMS derivative of vanillylpropionic acid (VPA) respectively for 38 control subjects (A) and 40 autistic patients (B). The x axis corresponds to the surface ratio of the amount of vanillylpropionic acid. The median values are represented as well as their 95% percentiles. The median value with autistic patients (B) is 0.04 and the median value for healthy controls (A) is 0.011. Data from chromatographic analyses show that the surface ratio, in relation to the inner standard, of the amount of said compound with autistic patients is from 0.014 to 0.053, and that it is from 0.003 to 0.022 with healthy controls. The Mann and Whitney test reveals a significant difference with an Ucal figure of 4.47.

FIG. 9:

FIG. 9 corresponds to the comparative bar chart pertaining to the amount of TMS derivative of dihydroxyphenylpropionic acid between 27 healthy controls (A), 27 autistic patients (B) and 18 non autistic patients suffering from intestinal dysbioses (C). The median values are represented as well as their 95% percentiles. The surface ratio of the amount of said compound with non autistic patients suffering from intestinal dysbioses is above that of autistic patients.

### EXPERIMENTAL PART

### Example 1

### Frequency and Amount of 3-methoxy-4-hydroxyphenyl-3-Hydroxy-Propionic Acid with Autistic Patients

A GC/MS analysis of the urine from 27 autistic patients allows one to identify an important peak with the same characteristics (retention time and mass spectrum) as those of 3-methoxy-4-hydroxyphenyl-3-hydroxy-propionic acid. This peak was quantified by integration of the area comprised under the peak of specific ionic fragments with a m/z ratio of 297 and 428. An analysis carried out on the urines of patients shows that the frequency of cases where the compound as described in the invention is detected in the urine is statistically different with autistic patients when compared with 27 healthy individuals.

The inner standard which is used is orthohydroxyphenylacetic acid, whose quantification is based upon fragmentation ions 296, 281, 252 and 164.

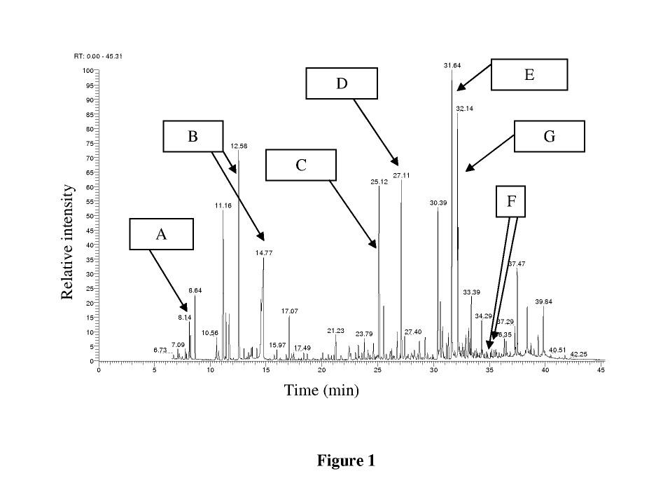

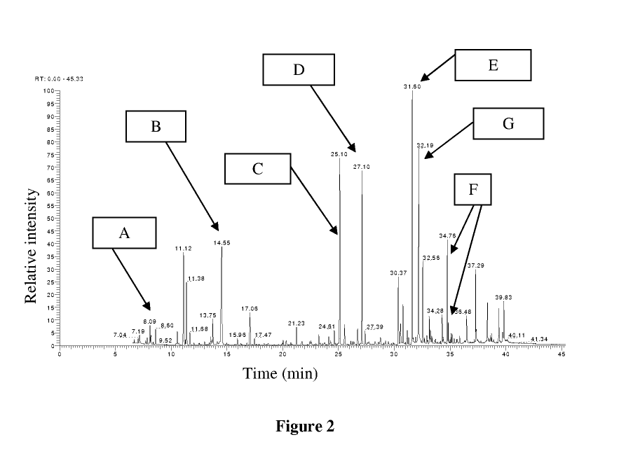

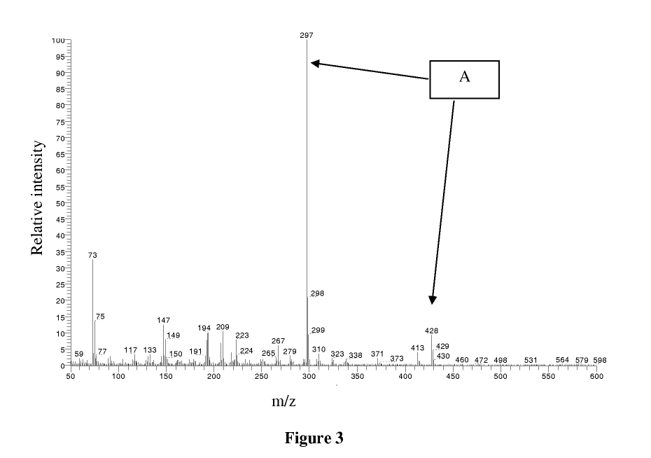

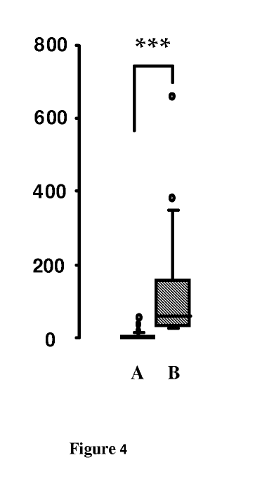

The results are reported in FIGS. 1-4.

### Example 2

### Frequency and Amount of 3-methoxy-4-hydroxyphenyl-3-Hydroxy-Propionic Acid with Patients Suffering from Other Psychiatric Disorders

A GC/MS analysis of the urine of 45 patients suffering from other psychiatric disorders allows one to identify an important peak having the same characteristics (retention time and mass spectrum) as 3-methoxy-4-hydroxyphenyl-3-hydroxy-propionic acid. This peak was quantified by integration of the area comprised under the peak of specific ionic fragments with a m/z ratio of 297 and 428. An analysis carried out on the urines of several patients shows that the frequency of cases where the compound as described in the invention is detected in the urine is statistically different with non autistic patients who suffer from psychiatric and/or psychomotor disorders when compared with 62 healthy individuals.

The inner standard which is used is orthohydroxyphenylacetic acid, whose quantification is based upon fragmentation ions 296, 281, 252 and 164.

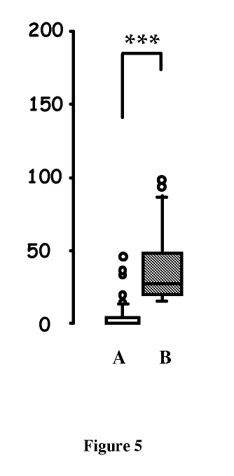

The results are reported in FIG. 5.

### Example 3

### Frequency and Amount of Vanillylglycine (VG) with Autistic Patients

The protocol which is used is the same as that of Example 1.

It has made it possible to identify an important peak with the same characteristics (retention time and mass spectrum) as those of vanillylglycine. This peak was quantified by integration of the area comprised under the peak of specific ionic fragments with a m/z ratio of 369, 354 and 223.

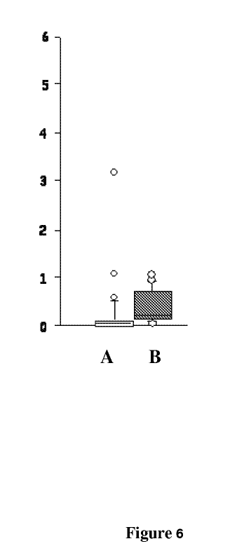

The result is reported in FIG. 6.

### Example 4

### Frequency and Amount of Vanillic Acid (VA) with Autistic Patients

The protocol which is used is the same as that of Example 1.

It has made it possible to identify an important peak with the same characteristics (retention time and mass spectrum) as those of vanillic acid (VA). This peak was quantified by integration of the area comprised under the peak of specific ionic fragments with a m/z ratio of 312, 297 and 267.

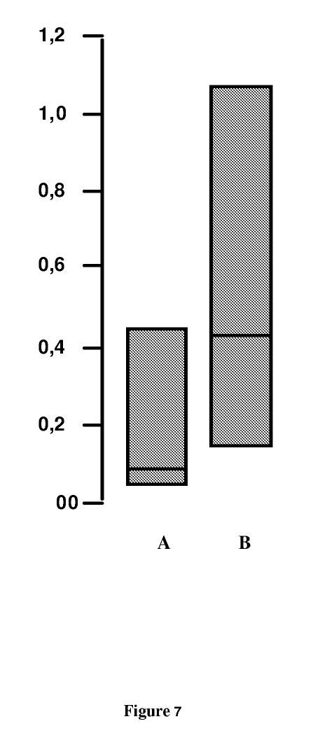

The result is reported in FIG. 7.

### Example 5

### Frequency and Amount of Vanillylpropionic Acid (VPA) with Autistic Patients

The protocol which is used is the same as that of Example 1.

It has made it possible to identify an important peak with the same characteristics (retention time and mass spectrum) as those of vanillylpropionic acid (VPA). This peak was quantified by integration of the area comprised under the peak of specific ionic fragments with a m/z ratio of 340, 310, and 325.

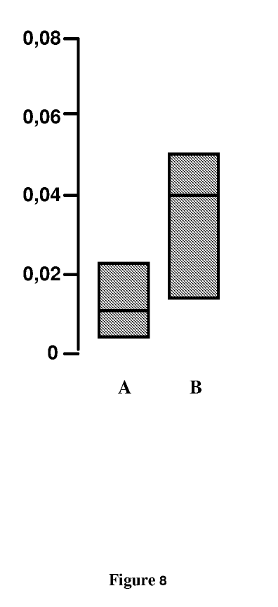

The result is reported in FIG. 8.

### Example 6

### Frequency and Amount of Dihydroxyphenylpropionic Acid with Autistic Patients and with Non Autistic Patients Suffering from Intestinal Dysbioses

The protocol which is used is the same as that of Example 1, comprising 18 patients suffering from dysbiosis, 27 autistic patients and 27 healthy individuals.

It has made it possible to identify an important peak with the same characteristics (retention time and mass spectrum) as those of dihydroxyphenylpropionic acid. This peak was quantified by integration of the area comprised under the peak of specific ionic fragments with a m/z ratio of 398, 267 and 179.

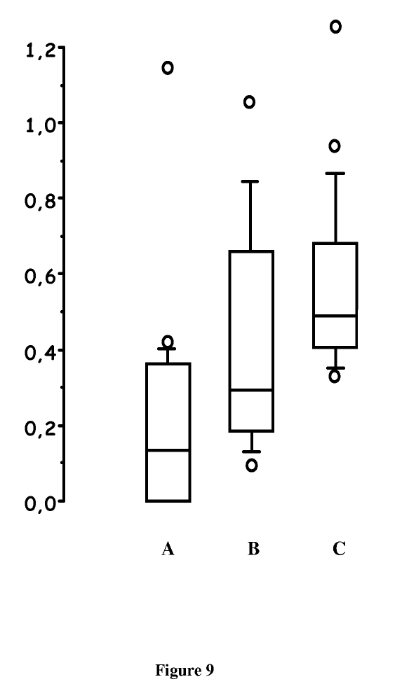

The results of FIG. 9 show that dihydroxyphenylpropionic acid allows one to diagnose dysbioses which are not associated with psychiatric disorders. In other words it does not seem to be a specific biological marker for the diagnosis of psychiatric disorders.

1.-21. (canceled)

22. A diagnostic method for psychiatric disorders and/or intestinal dysbioses comprising the administration in a patient in a need thereof, of at least one of the following Formula (I) compounds: 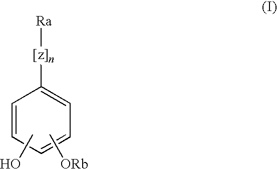
wherein
n=0 or 1,
Ra represents —CH₂OH, —COOH, —NR₁R₂, wherein R₁ and R₂ independently represent a hydrogen atom or a C₁-C₃ alkyl group,
Rb represents a C₁-C₃ alkyl group,
Z represents —CH₂—Y—, wherein Y represents a C₁-C₂ hydrocarbon chain, which may be substituted by an OH group, wherein said chain may comprise a —NH— and/or —CO— group,
wherein said compounds may be in the form of salts or, when at least one asymmetric carbon atom is present, in the form of isolated isomers or as a racemic mixture,
wherein said compounds differ from vanillyllactic acid, vanillylpyruvic acid and 3-methoxy-4-hydroxyphenylglycol,
as a physiological marker.

23. The method according to claim 22, comprising the administration in a patient in a need thereof of at least one of the following Formula (I′) compounds: 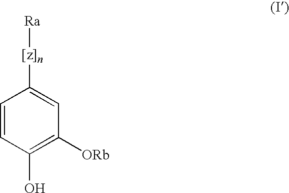
wherein
n=0 or 1,
Ra represents —CH₂OH, —COOH, —NR₁R₂, wherein R₁ and R₂ independently represent a hydrogen atom or a C₁-C₃ alkyl group,
Rb represents a C₁-C₃ alkyl group,
Z represents —CH₂—Y—, wherein Y represents a C₁-C₂ hydrocarbon chain, which may be substituted with an OH group, wherein said chain may comprise a —NH— and/or —CO— group,
wherein, as the case may be, said compounds are in the form of salts or, when at least one asymmetric carbon atom is present, in the form of isolated isomers or as a racemic mixture,
wherein said compounds differ from vanillyllactic acid, vanillylpyruvic acid and 3-methoxy-4-hydroxyphenylglycol,
as a physiological marker.

24. The method according to claim 22, comprising the administration in a patient in a need thereof of at least one of the following Formula (I) compounds 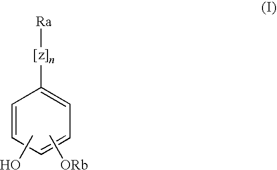
wherein
n=0 or 1,
Ra represents —COOH,
Rb represents a C₁-C₃ alkyl group,
Z represents —CH₂—Y—, wherein Y represent a C₁-C₂ hydrocarbon chain, which may be substituted with an OH group, wherein said chain may comprise a —NH— and/or —CO— group,
wherein said compounds may be in the form of salts or, when at least one asymmetric carbon atom is present, in the form of isolated isomers or as a racemic mixture,
wherein said compounds differ from vanillyllactic acid and vanillylpyruvic acid.

25. The method according to claim 22, comprising the administration in a patient in a need thereof of at least one of the following Formula (I) compounds: 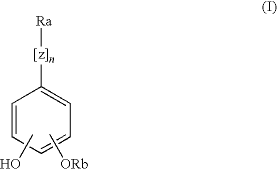
wherein
n=0 or 1,
Ra represents —COOH,
Rb represents a CH₃ group,
Z represents —CH₂—Y—, wherein Y represent a C₁-C₂ hydrocarbon chain, which may be substituted with an OH group, wherein said chain may comprise a —NH— and/or —CO— group,
wherein said compounds may be in the form of salts or, when at least one asymmetric carbon atom is present, in the form of isolated isomers or as a racemic mixture,
wherein said compounds differ from vanillyllactic acid and vanillylpyruvic acid.

26. The method according to claim 22, comprising the administration in a patient in a need thereof of at least one of the following Formula (II) compounds 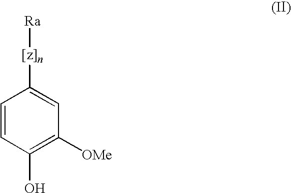
wherein n, Ra, and Z are as defined in claim 1.

27. The method according to claim 22, comprising the administration in a patient in a need thereof of at least one of the Formula (I) or (II) compounds, wherein:
n=0 or 1,
Ra represents —COOH,
Z represents —CH₂—CHOH—, —CH₂—CH₂—, —CO—NH—CH₂—,
wherein said compounds differ from vanillyllactic acid.

28. The method according to claim 22, comprising the administration in a patient in a need thereof of any of the Formula (I) or (II) compounds, wherein:
n=1,
Ra represents —COOH,
Rb represents —CH₃—,
Z represents —CH₂—CHOH—,
wherein said compounds differ from vanillyllactic acid.

29. The method according to claim 26, characterized in that the Formula (II) compound is chosen among one of the following compounds:

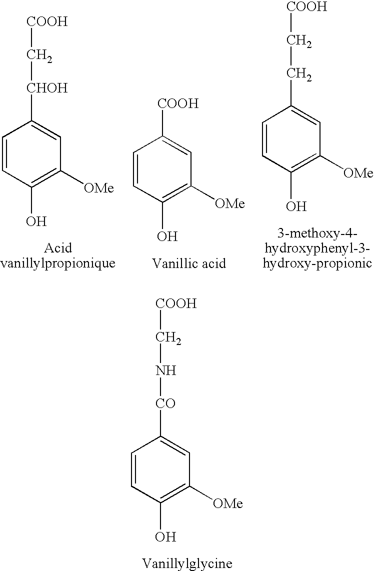

30. The method according to claim 26, characterized in that said compound corresponds to the following Formula:

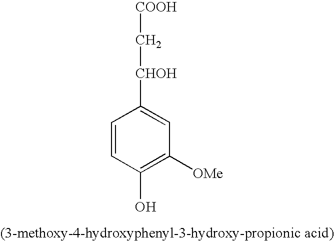

31. The method according to claim 26, characterized in that said compound corresponds to the following Formula:

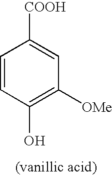

32. The method according to claim 22, characterized in that the psychiatric disorders belong to the group including autism, development retardations, neurodegenerative diseases such as Alzheimer's disease and Parkinson's disease.

33. The method according to claim 22, characterized in that said psychiatric disorder is autism.

34. The method according to claim 22, characterized in that said psychiatric disorder is autism, when linked to intestinal dysbiosis.

35. A method for the in vitro or ex vivo diagnosis of psychiatric disorders and/or intestinal dysbioses, characterized in that it includes a step for the determination of a variation in the amount of at least one of the following Formula (I) compounds: 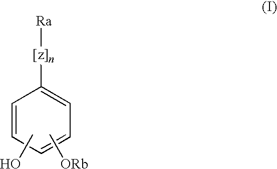
wherein:
n=0 or 1,
Ra represents —CH₂OH, —COOH, —NR₁R₂, wherein R₁ and
R₂ independently represent a hydrogen atom or a C₁-C₃ alkyl group,
Rb represents a C₁-C₃ alkyl group,
Z represents —CH₂—Y—, wherein Y represents a C₁-C₂ hydrocarbon chain, which may be substituted by an OH group, wherein said chain may comprise a —NH— and/or —CO— group,
wherein said compounds may be in the form of salts or, when at least one asymmetric carbon atom is present, in the form of isolated isomers or as a racemic mixture,
wherein said compounds differ from vanillyllactic acid, vanillylpyruvic acid and 3-methoxy-4-hydroxyphenylglycol,
as present in a biological sample which is taken from a patient, in relation to the amount of this (these) same compound(s) as present in a biological sample which is taken from a healthy individual.

36. The method according to claim 35, characterized in that it includes a step for the determination of a variation in the amount of at least one of the following Formula (I) compounds: 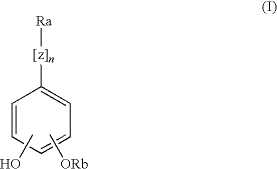
wherein:
n=0 or 1,
Ra represents —CH₂OH, —COOH, —NR₁R₂, wherein R₁ and R₂ independently represent a hydrogen atom or a C₁-C₃ alkyl group,
Rb represents a CH₃ group,
Z represents —CH₂—Y—, wherein Y represents a C₁-C₂ hydrocarbon chain, which may be substituted by an OH group, wherein said chain may comprise a —NH— and/or —CO— group,
wherein said compounds may be in the form of salts or, when at least one asymmetric carbon atom is present, in the form of isolated isomers or as a racemic mixture,
wherein said compounds differ from vanillyllactic acid, vanillylpyruvic acid and 3-methoxy-4-hydroxyphenylglycol,
as present in a biological sample which is taken from a patient, in relation to the amount of this (these) same compound(s) as present in a biological sample which is taken from a healthy individual.

37. The method according to claim 35, characterized in that the determination by an appropriate method of an increase in the amount of at least one of the compounds as previously defined as present in a biological sample which is taken from a patient, in relation to the amount of this (these) same compound(s) as present in a biological sample which is taken from a healthy individual, corresponds to the diagnosis of autism and/or intestinal dysbioses.

38. The method according to claim 35, characterized in that the amount of at least one of the compounds as previously defined, as present in a biological sample which is taken from a patient, and that the amount of this (these) same compound(s) as present in a biological sample which is taken from a healthy individual, is measured by chromatography, by spectrophotometry, by an immunological method, notably by an ELISA assay, or by an immuno-nephelemetric method.

39. The method according to claim 35, characterized in that the determination of an increase in the amount of said compound of the Formula: 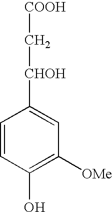
as present in a biological sample which is taken from a patient, in relation to the amount of this same compound as present in a biological sample which is taken from a healthy individual, corresponds to the diagnosis of autism and/or intestinal dysbioses.

40. The method for the in vitro or ex vivo diagnosis according to claim 35, characterized in that the biological sample is chosen among urine, blood, saliva, the cerebrospinal fluid or stools.

41. The method according to claim 35, characterized in that the amount of at least one of the compounds as previously defined, as present in a urine sample which is taken from a patient, is superior or equal to 20 micrograms per milligram of creatinine.

42. The method according to claim 35, characterized in that the amount of 3-methoxy-4-hydroxyphenyl-3-hydroxy-propionic acid, as present in a urine sample which is taken from a patient, is superior or equal to 20 micrograms per milligram of creatinine.

43. The method according to claim 35, characterized in that the amount of 3-methoxy-4-hydroxyphenyl-3-hydroxy-propionic acid, as present in a urine sample which is taken from a patient, is from 20 to 180 micrograms per milligram of creatinine.

44. A diagnostic kit for psychiatric disorders and/or intestinal dysbioses, comprising:
one or several reagents, which may detect the presence, in a biological sample, of at least one of the following Formula (I) compounds: 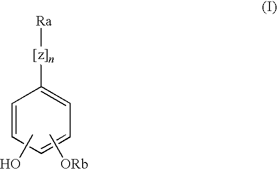
wherein:
n=0 or 1,
Ra represents —CH₂OH, —COOH, —NR₁R₂, wherein R₁ and R₂ independently represent a hydrogen atom or a C₁-C₃ alkyl group,
Rb represents a C₁-C₃ alkyl group,
Z represents —CH₂—Y—, wherein Y represents a C₁-C₂ hydrocarbon chain, which may be substituted by an OH group, wherein said chain may comprise a —NH— and/or —CO— group,
wherein said compounds may be in the form of salts or, when at least one asymmetric carbon atom is present, in the form of isolated isomers or as a racemic mixture,
wherein said compounds differ from vanillyllactic acid, vanillylpyruvic acid and 3-methoxy-4-hydroxyphenylglycol,
as the case may be, a control sample containing at least one of the compounds, in a predetermined amount which is indicative of a positive diagnosis for psychiatric disorders and/or intestinal dysbioses,
as the case may be, a control sample containing at least one of the compounds in a predetermined amount which is indicative of a negative diagnosis for psychiatric disorders and/or intestinal dysbioses.

45. A diagnostic kit according to claim 44,
wherein said reagents are antibodies against at least one of the following Formula (I) compounds: 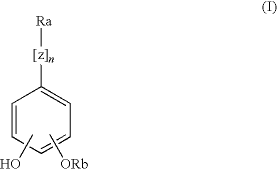
wherein:
n=0 or 1,
Ra represents —CH₂OH, —COOH, —NR₁R₂, wherein R₁ and R₂ independently represent a hydrogen atom or a C₁-C₃ alkyl group,
Rb represents a C₁-C₃ alkyl group,
Z represents —CH₂—Y—, wherein Y represents a C₁-C₂ hydrocarbon chain, which may be substituted by an OH group, wherein said chain may comprise a —NH— and/or —CO— group,
wherein said compounds may be in the form of salts or, when at least one asymmetric carbon atom is present, in the form of isolated isomers or as a racemic mixture,
wherein said compounds differ from vanillyllactic acid, vanillylpyruvic acid and 3-methoxy-4-hydroxyphenylglycol,
which may detect the presence, in a biological sample, of at least one of the above defined Formula (I) compounds.

46. A diagnostic kit according to claim 45, wherein in Formula (I) compounds Rb represents a CH₃ group,

47. Monoclonal or polyclonal antibodies against at least one of the following Formula (I) compounds: 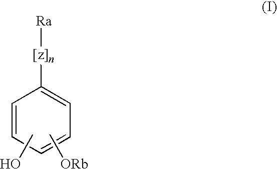
wherein:
n=0 or 1,
Ra represents —CH₂OH, —COOH, —NR₁R₂, wherein R₁ and R₂ independently represent a hydrogen atom or a C₁-C₃ alkyl group,
Rb represents a C₁-C₃ alkyl group,
Z represents —CH₂—Y—, wherein Y represents a C₁-C₂ hydrocarbon chain, which may be substituted by an OH group, wherein said chain may comprise a —NH— and/or —CO— group,
wherein said compounds may be in the form of salts or, when at least one asymmetric carbon atom is present, in the form of isolated isomers or as a racemic mixture,
wherein said compounds differ from vanillyllactic acid, vanillylpyruvic acid and 3-methoxy-4-hydroxyphenylglycol,

48. Monoclonal or polyclonal antibodies according to claims 47, wherein antibodies are directed against 3-methoxy-4-hydroxyphenyl-3-hydroxy-propionic acid.

49. Hybridoma which may produce monoclonal antibodies as defined according to claim 47.
